(19) United States
(12) Patent Application Publication
(10) Pub. No.: US 2026/0166024 A1
(43) Pub. Date: Jun. 18, 2026

(54) AN AURORA A INHIBITOR FOR USE IN TREATMENTS OF CANCERS

(71) Applicant: JS INNOPHARM (SUZHOU) LTD., Suzhou (CN)

(72) Inventors: Ao LI, Beijing (CN); Jintao ZHANG, Naperville, IL (US)

(21) Appl. No.: 19/124,902

(22) PCT Filed: Oct. 23, 2023
(86) PCT No.: PCT/CN2023/125864

§ 371 Date: Apr. 28, 2025

(30) Foreign Application Priority Data
Oct. 26, 2022 (WO) PCT/CN2022/127664

(51) Int. Cl. A61K 31/4545; A61P 35/00; C07D 401/14

(52) U.S. Cl. CPC A61K 31/4545; A61P 35/00; C07D 401/14

(57) ABSTRACT

Disclosed herein is a method for treating a cancer in a subject in need thereof, comprising identifying the cancer to be a BRCA mutation cancer or a BRCA wild type cancer having a homologous recombination deficiency; and administering to the subject a therapeutically effective amount of an Aurora A inhibitor, or a pharmaceutically acceptable salt, hydrate, solvate, amorphous solid, or polymorph thereof.

### TECHNICAL FIELD

The present disclosure relates to an Aurora A inhibitor 1-(2,3-dichlorobenzoyl)-4-[5-fluoro-6-(5-methyl-1H-pyrazol-3-ylamino)pyridin-2-yl]methyl-4-piperidinecarboxylic acid or a pharmaceutical acceptable salt thereof for use in the treatment of cancers, specifically, the cancer having a BRCA mutation and/or a homologous recombination deficiency.

### TECHNICAL BACKGROUND

Genomic instability is a hallmark of cancer cells (Hanahan and Weinberg, Cell, 144 (5): 646-74 (2011)). To maintain genomic stability and ensure high-fidelity transmission of genetic information, cells have evolved a complex mechanism to repair DNA double-strand breaks (DSBs), the most deleterious DNA lesions, in an error-free manner through homologous recombination (HR) (Moynahan and Jasin, Nat. Rev. Mol. Cell Biol., 11 (3): 196-207 (2010); San Filippo J et al., Annu. Rev. Biochem., 77:229-57 (2008)). Therefore, cells deficient in HR show retarded growth and exhibit higher level of genetic instability. It is believed that genetic instability due to loss of HR repair in human cancers significantly contributes to the development of cancer in these cells.

BRCA1 and/or BRCA2 (referred to collectively as “BRCA”) genes are crucial tumor suppressors for HR repair. Proteins encoded by BRCA gene involve in the repair of DNA double-strand breaks and cell growth and prevent abnormal cell division leading to the developments of tumors (Farmer H et al., Nature, 434:917-921 (2005)). Therefore, the BRCA gene mutation means that the tumor suppressor gene “brakes” are out of control and cannot effectively inhibit the proliferation of tumor cells. BRCA gene mutations and other HR repair defects are closely related to the development of ovarian, prostate, breast, and pancreatic cancers.

Aurora kinases are a family of serine/threonine kinases and are key regulators of mitosis. There are three human homologs of Aurora kinases, A, B, and C. Aurora A is involved in, e.g., formation and maturation of a centrosome, spindle kinetics and chromosome alignment in the mitotic phase (M phase) of the cell cycle, and regulation of a process of mitotic division (WO 2013/129443). Up to the present, overexpression and/or amplification of Aurora A have been confirmed in a wide variety of carcinomas (US 2015-0065479). Furthermore, inhibition of Aurora A kinase in a tumor cell induces arrest of mitotic division and apoptosis. Thus, Aurora A becomes a promising cancer drug target.

Recently, the function of Aurora A kinase in mediating DNA repair and the DNA damage response (DDR) has been increasingly studied. These studies show that Aurora kinase A (AURKA) may mediate chromosomal instability in tumor cells by regulating error-prone non-homologous end joining (NHEJ) DNA repair. In vitro studies in breast cancer cells revealed that AURKA overexpression diminished recruitment of RAD51 to sites of DSBs, which disrupted repair of DNA damage through the high-fidelity homologous recombination (HR)-dependent mechanism, thereby favoring the NHEJ pathway (Sourisseau T et al., EMBO Mol. Med., 2:130-42 (2010)). The error-prone NHEJ results in chromosomal translocations and rearrangements, leading to a genomic instability and thus the generation of tumors (Guirouilh-Barbat J et al., Mol. Cell., 14:611-23 (2004)).

The recent translational medicine research demonstrates that Aurora A kinase inhibition is synthetic lethal with loss of the RB1 tumor suppressor gene, and Aurora A kinase inhibitors are able to inhibit the formation of resistance mechanisms in third-generation EGFR inhibitors (Gong X et al., Cancer Discov., 9 (2): 248-263 (2019); Shah K N et al., Nat. Med., 25:111-118 (2019)).

Therefore, there is still a need to expand the indications for Aurora A kinase inhibitors.

### SUMMARY OF THE DISCLOSURE

Provided in the present disclosure is a method for treating a cancer in a subject in need thereof, comprising: a) identifying the cancer to be a BRCA mutation cancer or a BRCA wild type cancer with a homologous recombination deficiency (HRD); and b) administering to the subject a therapeutically effective amount of an Aurora A inhibitor or a pharmaceutically acceptable salt, hydrate, solvate, amorphous solid, or polymorph thereof. The inventors of present disclosure surprisingly found that the Aurora A inhibitor is effective in inhibiting proliferations of tumor cells having BRCA mutations or proliferations of BRCA wild type tumor cells with a homologous recombination deficiency. The inventors of the present disclosure have also found that, in some cases, the Aurora A inhibitors achieve a better anti-tumor efficacy than the PARP inhibitors that have been approved in the treatment of BRCA mutation cancer.

In one aspect of the present disclosure, the cancer is a BRCA mutant breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, or gastric cancer. In some embodiments, the cancer is a BRCA mutant breast cancer, ovarian cancer, prostate cancer, colorectal cancer, head and neck cancer, liver cancer, non-small cell lung cancer (NSCLC), small cell lung cancer (SCLC) or gastric cancer.

In one aspect of the present disclosure, the BRCA mutation cancer is a BRCA2 mutant cancer.

In one aspect of the present disclosure, the cancer is a BRCA2 mutant cancer having a homologous recombination deficiency.

In one aspect of the present disclosure, the cancer is a BRCA2 mutant breast cancer, prostate cancer, colorectal cancer, small cell lung cancer or gastric cancer.

In one aspect of the present disclosure, the cancer is a BRCA2 mutant breast cancer.

In one aspect of the present disclosure, the cancer is a BRCA2 mutant prostate cancer.

In one aspect of the present disclosure, the cancer is a BRCA2 mutant small cell lung cancer.

In one aspect of the present disclosure, the cancer is a BRCA2 mutant gastric cancer.

In one aspect of the present disclosure, the cancer is a BRCA2 mutant breast cancer having a homologous recombination deficiency.

In one aspect of the present disclosure, the cancer is a BRCA1 mutant cancer.

In one aspect of the present disclosure, the cancer is a BRCA1 mutant breast cancer, head and neck cancer, or liver cancer.

In one aspect of the present disclosure, the cancer is a BRCA1 mutant breast cancer.

In one aspect of the present disclosure, the cancer is a BRCA1 mutant head and neck cancer.

In one aspect of the present disclosure, the cancer is a BRCA1 mutant liver cancer.

In one aspect of the present disclosure, the cancer is a BRCA1 and BRCA2 mutant cancer.

In one aspect of the present disclosure, the cancer is a BRCA1 and BRCA2 mutant ovarian cancer, prostate cancer, or small cell lung cancer.

In one aspect of the present disclosure, the cancer is a BRCA1 and BRCA2 mutant ovarian cancer.

In one aspect of the present disclosure, the cancer is a BRCA1 and BRCA2 mutant small cell lung cancer.

In one aspect of the present disclosure, the cancer is a BRCA wild type cancer having a homologous recombination deficiency.

In one aspect of the present disclosure, the cancer is a BRCA wild type breast cancer having a homologous recombination deficiency.

In one aspect of the present disclosure, the Aurora A inhibitor is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof. In some embodiments, the Aurora A inhibitor is provided as a free base. In some embodiments, the Aurora A inhibitor is provided as an acid addition salt. In one preferred embodiment, the Aurora A inhibitor is a monohydrochloride salt that is 1-(2,3-dichlorobenzoyl)-4-[5-fluoro-6-(5-methyl-1H-pyrazol-3-ylamino)pyridin-2-yl]methyl-4-piperidine carboxylic acid monohydrochloride (which is also known as TAS-119 or VIC-1911).

In one aspect of the present disclosure, the Aurora A inhibitor is orally administered twice per day, wherein the Aurora A inhibitor's unit dose at each administration ranges from 15 mg/kg to 200 mg/kg, preferably from 20 mg/kg to 150 mg/kg, or more preferably from 60 mg/kg to 120 mg/kg. In some embodiments, the Aurora A inhibitor is orally administered about every 12 hours.

In one aspect of the present disclosure, the Aurora A inhibitor further comprises a pharmaceutically acceptable carrier, diluent, or excipient.

In another aspect, the present disclosure relates to the use of an Aurora A inhibitor in the treatment of a BRCA mutation cancer or a BRCA wild type cancer with an HRD.

In some aspects of the present disclosure, the Aurora A inhibitor is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof.

In a further aspect, the present disclosure relates to a compound which is 1-(2,3-dichlorobenzoyl)-4-[5-fluoro-6-(5-methyl-1H-pyrazol-3-ylamino)pyridin-2-yl]methyl-4-piperidine carboxylic acid, for use in the preparation of a medicament for treating a BRCA mutant cancer or a BRCA wild type cancer having an HRD.

In a further aspect, the present disclosure relates to a compound, which is 1-(2,3-dichlorobenzoyl)-4-[5-fluoro-6-(5-methyl-1H-pyrazol-3-ylamino)pyridin-2-yl]methyl-4-piperidine carboxylic acid, for use in treating a BRCA mutant cancer or a BRCA wild type cancer having an HRD.

Additional aspects and advantages of the disclosure will be set forth, in part, in the description that follows, and will flow from the description, or can be learned by practice of the disclosure. The aspects and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF FIGURES

FIGS. 1A and 1B show the anti-tumor activity and tolerability of VIC-1911 at different unit doses (15 mg/kg BID, 30 mg/kg BID, 60 mg/kg BID, 120 mg/kg BID) in mice using the MDA-MB-436 BRCA1 mutant cell-derived xenografts (CDX) breast cancer model.

FIGS. 2A and 2B show the anti-tumor activity and tolerability of VIC-1911 in mice using the DU145 BRCA1 and BRCA2 mutant CDX prostate cancer model.

FIGS. 3A and 3B show the anti-tumor activity and tolerability of VIC-1911 relative to PARP inhibitor Olaparib and Androgen Receptor antagonist Enzalutamide in mice using the 22RV1 BRCA2 mutant CDX prostate cancer model.

FIGS. 4A and 4B show the anti-tumor activity and tolerability of VIC-1911 in mice using the BR-05-0014E BRCA2 mutant patent-derived xenografts (PDX) triple-negative breast cancer model, in which the cancer cell is evaluated with an HRD score of 68.93.

FIGS. 5A and 5B show the anti-tumor activity and tolerability of VIC-1911 in mice using the BR-05-0028 BRCA1 mutant PDX breast cancer model.

FIGS. 6A and 6B show the anti-tumor activity and tolerability of VIC-1911 in mice using the OV-10-0060 BRCA2 mutant PDX ovarian cancer model.

FIGS. 7A and 7B show the anti-tumor activity and tolerability of VIC-1911 in mice using the OV-10-0079 BRCA1 and BRCA2 mutant PDX ovarian cancer model.

FIGS. 8A and 8B show the anti-tumor activity and tolerability of VIC-1911 in mice using the HN-13-0032 BRCA1 mutant PDX head and neck cancer model.

FIGS. 9A and 9B show the anti-tumor activity and tolerability of VIC-1911 in mice using the LU-01-1388 BRCA1 and BRCA2 mutant PDX small cell lung cancer model.

FIGS. 10A and 10B show the anti-tumor activity and tolerability of VIC-1911 in mice using the LU-01-1116 BRCA2 mutant PDX small cell lung cancer model.

FIGS. 11A and 11B show the anti-tumor activity and tolerability of VIC-1911 in mice using the ST-02-0360 BRCA2 mutant PDX gastric cancer model.

FIGS. 12A and 12B show the anti-tumor activity and tolerability of VIC-1911 in comparison to the PARP inhibitor Olaparib in mice using the BR-05-0020E BRCA wild type PDX breast cancer model, in which the tumor sample is evaluated with an HRD score of 64.83.

FIGS. 13A and 13B show the anti-tumor activity and tolerability of VIC-1911 relative to the PARP inhibitor Olaparib in mice using the LI-03-0014 BRCA1 mutant PDX liver cancer model.

FIGS. 14A and 14B show the anti-tumor activity and tolerability of VIC-1911 relative to the PARP inhibitor Olaparib in mice using the CO-04-0003 BRCA2 mutant PDX colorectal cancer model.

### DETAILED DESCRIPTION OF THE DISCLOSURE

One aspect of the present disclosure is based on the use of an Aurora A kinase inhibitor in the treatment of a BRCA mutant cancer or a BRCA wild type cancer having a homologous recombination deficiency.

The inventors of the present disclosure surprisingly found that an Aurora A inhibitor, particularly, 1-(2,3-dichlorobenzoyl)-4-[5-fluoro-6-(5-methyl-1H-pyrazol-3-ylamino)pyridin-2-yl]methyl-4-piperidine carboxylic acid, is effective to inhibit proliferations of tumor cells having BRCA mutations or proliferations of BRCA wild type tumor cells with a homologous recombination deficiency. In some cases, the Aurora A inhibitors achieve a better anti-tumor efficacy than the PARP inhibitors that have been approved in the treatment of BRCA mutation cancer.

In some aspects, the Aurora A inhibitor disclosed herein is in a therapeutically effective amount sufficient to produce a therapeutic effect comprising: (i) a reduction in size of a tumor, (ii) a reduction of tumor regression, and/or (iii) a reduction or inhibition of cancer tumor growth. In some aspects, the Aurora A inhibitor disclosed herein can delay, reduce, or prevent rebounding (rapid re-growth) of a tumor.

The Aurora A inhibitors disclosed herein are well tolerated (lack of toxicity) in the treatment of a BRCA mutant cancer or a BRCA wild type cancer having a homologous recombination deficiency.

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents, and other references mentioned herein are incorporated by reference in their entireties for all purposes as if each individual publication or patent application were specifically and individually indicated to be incorporated by reference.

Although methods and materials similar or equivalent to those described herein can be used in the practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

It is understood that embodiments described herein include “consisting” and/or “consisting essentially of” embodiments. As used herein, the singular form “a,” “an,” and “the” includes plural references unless indicated otherwise. Use of the term “or” herein is not meant to imply that alternatives are mutually exclusive.

In this disclosure, the use of “or” means “and/or” unless expressly stated or understood by one skilled in the art. In the context of a multiple dependent claim, the use of “or” refers back to more than one preceding independent or dependent claim.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, a description referring to “about X” includes a description of “X.”

As used herein, “treatment” is an approach for obtaining beneficial or desired clinical results. “Treatment” as used herein, covers any administration or application of a therapeutic agent for disease in a mammal, including a human. For purposes of the presentdisclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delaying or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term “treatment” does not require one-hundred percent removal of all aspects of the disease or disorder.

In the context of cancer, the term “treating” as used herein includes, but is not limited to, inhibiting growth of cancer cells, inhibiting replication of cancer cells, lessening of overall tumor burden, and delaying, halting, or slowing tumor growth, progression, or metastasis.

As used herein, “delaying” means to defer, hinder, slow, retard, stabilize, suppress and/or postpone development or progression of the disease (such as cancer). This delay can be of varying lengths of time, depending on the history of the disease and/or individual being treated.

As used herein “therapeutically effective amount” of a substance refers to an amount of said substance that is effective, at dosages and for periods of time necessary, to achieve the desired therapeutic effect. A therapeutically effective amount can vary according to factors such as the disease state, age, sex, and weight of the individual, and the ability of the substance to elicit a desired response in the individual. A therapeutically effective amount is also one in which any toxic or detrimental effects of the substance are outweighed by the therapeutically beneficial effects. A therapeutically effective amount can be delivered in one or more administrations.

As used herein, a “homologous recombination deficiency” or “homologous recombination defect” is defined as a deficiency or defect in homologous recombination with a “homologous recombination deficiency score” or “HRD score” of no less than 42. The HRD score is evaluated according to the method as described in Telli M L et al., Clin. Cancer Res., 22 (15): 3764-73 (2016), which is herein incorporated by reference in its entirety.

The terms “administer,” “administering,” “administration,” and the like, as used herein, refer to methods that can be used to enable delivery of the therapeutic agent to the desired site of biological action. Administration techniques can be employed with the existing agents and methods. Administration of two or more therapeutic agents includes simultaneous (concurrent) and consecutive administration in any order.

The term “pharmaceutical composition” as used herein refers to a preparation which is in such form as to permit the biological activity of the active ingredient(s) to be effective, and which contains no additional components which are unacceptably toxic to a subject to which the formulation would be administered. Such formulations may be sterile.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salt” as used herein refers to a salt form of a compound. A salt form of a compound is typically a crystalline form comprising the compound and one or more salt formers in which the compound and salt former molecules are in an ionized state and arranged in the same crystal lattice.

The term “solvate” as used herein refers to a crystalline form of a compound wherein molecules of a solvent or solvents are incorporated into the crystal lattice. The ratio of compound molecules to solvent molecules in a solvate may be stoichiometric or nonstoichiometric.

The term “hydrate” as used herein refers to a solvate in which the solvent incorporated into the crystal lattice is water.

The term “polymorph” as used herein refers to a crystalline form of a compound having a particular molecular packing arrangement in the crystal lattice. Crystalline forms can be identified and distinguished from each other by one or more characterization techniques including, for example, X-ray powder diffraction (XRPD), single crystal X-ray diffraction, and ¹³C solid state nuclear magnetic resonance (¹³C SSNMR).

The term “amorphous solid” as used herein refers to a solid material having no long-range order in the position of its molecules. Amorphous solids are typically supercooled liquids in which the molecules are arranged in a random manner so that there is no well-defined arrangement, e.g., molecular packing, and no long-range order.

The term “disease” or “condition” or “disorder” as used herein refers to a condition where treatment is needed and/or desired and denotes disturbances and/or anomalies that as a rule are regarded as being pathological conditions or functions, and that can manifest themselves in the form of particular signs, symptoms, and/or malfunctions. As demonstrated below, the AURKA inhibitors disclosed herein can be used in treating diseases and conditions, such as proliferative diseases, wherein inhibition of AURKA provides a benefit.

The term “AURKA” as used herein refers to the Aurora A kinase. In humans, the Aurora A kinase is encoded by the AURKA gene. Aurora A kinase is a member of a family of serine/threonine kinases. Aurora A kinase is one of three human homologs of Aurora kinases, Aurora A, B, and C.

The term “PARP” or “PARP protein” as used herein refers to one or more of the Poly (ADP-ribose) polymerase family of enzymes. The family includes enzymes that have the ability to catalyze the transfer of ADP-ribose to target proteins (poly ADP-ribosylation). There are at least 18 members of the PARP family that are encoded by different genes, and share homology in a conserved catalytic domain, including PARP-1, PARP-2, and PARP-3.

The terms “reduction” or “reduce” or “inhibition” or “inhibit” as used herein refer to a decrease or cessation of any phenotypic characteristic or to the decrease or cessation in the incidence, degree, or likelihood of that characteristic. As used herein, to “reduce” or “inhibit” is to decrease, reduce, or arrest an activity, function, and/or amount as compared to a reference. In some embodiments, to “reduce” or “inhibit” a characteristic meansto cause an overall decrease of 20% or greater of that characteristic. In some embodiments, to “reduce” or “inhibit” a characteristic means to cause an overall decrease of 50% or greater of that characteristic. In some embodiments, to “reduce” or “inhibit” a characteristic means to cause an overall decrease of 75%, 85%, 90%, 95%, or greater of that characteristic. In some embodiments, the amount noted above is inhibited or decreased over a period of time, relative to a control over the same period of time.

The terms “individual” and “subject” are used interchangeably herein to refer to an animal, for example, a mammal, such as a human. In some instances, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some instances, the subject to receive the treatment can be a patient, designating the fact that the subject has been identified as having a disorder of relevance to the treatment, or being at particular risk of contracting the disorder.

As used herein, the terms “cancer” and “tumor” refer to or describe the physiological condition in mammals in which a population of cells are characterized by unregulated cell growth. The terms encompass solid and hematological/lymphatic cancers. Examples of cancer include, but are not limited to, DNA damage repair pathway deficient cancers. Additional examples of cancer include, but are not limited to, breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, and gastric cancer. The cancer can be BRCA1 or BRCA2 wild type. The cancer can also be BRCA1 or BRCA2 mutant.

As used herein, the term “mutation” or “mutant” indicates any modification of a nucleic acid and/or polypeptide which results in an altered nucleic acid or polypeptide. The term “mutation” or “mutant” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

### AURKA Inhibitor

In some aspects, the Aurora A kinase inhibitor disclosed herein comprises a compound of Formula (I):

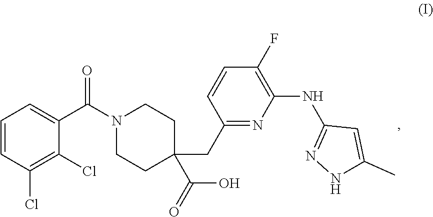 [SMILES: Cc1cc(Nc2nc(CC3(C(=O)O)CCN(C(=O)c4cccc(Cl)c4Cl)CC3)ccc2F)n[nH]1]

or a pharmaceutically acceptable salt, hydrate, solvate, amorphous solid, or polymorph thereof.

The chemical name for the AURKA inhibitor of Formula (I) is 1-(2,3-dichlorobenzoyl)-4-((5-fluoro-6-(5-methyl-1H-pyrazol-3-ylamino)pyridin-2-yl)methyl) piperidine-4-carboxylic acid, as described in PCT International Application Publication No. WO 2013/129443, which is herein incorporated by reference in its entirely.

The AURKA inhibitor, or pharmaceutically acceptable salt thereof, will normally be administered to a warm-blooded animal at a unit dose, for example, ranging from about 15 mg/kg to 200 mg/kg of active ingredient. The AURKA inhibitor can be orally administrated at a unit dose of 15 mg/kg, 20 mg/kg, 50 mg/kg, 80 mg/kg, 100 mg/kg, 120 mg/kg, 150 mg/kg or 200 mg/kg twice a day. For example, the AURKA inhibitor can be taken twice a day at a unit dose ranging from 15 mg/kg to 200 mg/kg, 15 mg/kg to 180 mg/kg, 20 mg/kg to 150 mg/kg, 30 mg/kg to 140 mg/kg, 50 mg/kg to 130 mg/kg, or 60 mg/kg to 120 mg/kg. However, the daily dose will necessarily be varied depending upon the host treated, the particular route of administration, and the severity of the illness being treated. Accordingly, the optimum dosage may be determined by the practitioner who is treating any particular patient.

In various aspects, the AURKA inhibitors reduce the level of AURKA protein and/or inhibit or reduce at least one biological activity of AURKA protein.

In some aspects, the AURKA inhibitors competitively bind to the hydrophobic ATP binding pocket of AURKA protein to inhibit the bioactivity of AURKA protein.

Any suitable assay in the art can be used to determine an activity, detect an outcome or effect, or determine efficacy. See, e.g. WO 2013/129443 that is herein incorporated by reference in its entirety.

### PARP Inhibitor

In various aspects, the PARP inhibitors disclosed herein reduce the level of one or more PARP proteins and/or inhibit or reduce at least one biological activity of one or more PARP proteins.

PARP inhibitors include, for example, olaparib (Lynparza®), rucaparib (Rubraca®), niraparib (Zejula®), talazoparib (Talzenna®), pamiparib (Partruvix®), and fuzoloparib.

In another aspect, the PARP inhibitor is olaparib (Lynparza®). The chemical name is 4-[(3-{[4-(cyclopropylcarbonyl) piperazin-1-yl]carbonyl}-4-fluorophenyl)-methyl]phthalazin-1 (2H)-one. The molecular formula is C₂₄H₂₃FN₄O₃, and the molecular weight is 434.5 g/mol.

Olaparib is an inhibitor of poly (ADP-ribose) polymerase (PARP) enzymes, including PARP-1, PARP-2, and PARP-3. Olaparib has been shown to inhibit growth of select tumor cell lines in vitro and decrease tumor growth in mouse xenograft models of human cancer, both as monotherapy or following platinum-based chemotherapy. Increased cytotoxicity and anti-tumor activity following treatment with olaparib were noted in cell lines and mouse tumor models with deficiencies in BRCA and non-BRCA proteins involved in the homologous recombination repair (HRR) of DNA damage and correlated with platinum response. In vitro studies have shown that olaparib-induced cytotoxicity may involve inhibition of PARP enzymatic activity and increased formation of PARP-DNA complexes, resulting in DNA damage and cancer cell death.

The PARP inhibitor, or pharmaceutically acceptable salt thereof, will normally be administered to a warm-blooded animal at a unit dose, for example, ranging from about 0.5 mg to 1 g of active ingredient. The PARP inhibitors can be orally administrated at a unit dose of 0.5 mg, 5 mg, 10 mg, 15 mg, 30 mg, 50 mg, 100 mg, 120 mg, or 250 mg once a day. For example, PARP inhibitors can be taken once a day at a dose ranging from 0.5 mg to 120 mg, 5 mg to 120 mg, 20 mg to 120 mg, 50 mg to 120 mg, 50 mg to 100 mg, or 70 mg to 100 mg. However, the daily dose will necessarily be varied depending upon the host treated, the particular route of administration, and the severity of the illness being treated. Accordingly, the optimum dosage may be determined by the practitioner who is treating any particular patient.

The present disclosure provides compounds that are active in inhibiting the activity of PARP. Any suitable assay in the art can be used to determine an activity, detect an outcome or effect, or determine efficacy. See, e.g., Dillon K J et al., J. Biomol. Screen., 8 (3): 347-352 (2003); U.S. Pat. No. 9,566,276.

### Androgen Receptor Antagonist

In various aspects, the androgen receptor antagonists of the present disclosure block the binding of androgen to the androgen receptor. The androgen receptor antagonists include, for example, Apalutamide, Enzalutamide, and darolutamide.

In another aspect, the androgen receptor antagonist is Enzalutamide, the chemical name of which is N-methyl-4-[3-(4-cyano-3-trifluoromethylphenyl)-5,5-dimethyl-4-oxo-2-thioxoimidazolidin-1-yl]-2-fluorobenzamide. The molecular formula is C₂₁H₁₆F₄N₄O₂S, and the molecular weight is 464.4 g/mol.

Methods of Identifying a BRCA mutant cancer or an HRD BRCA wild type (BRCA^{Wt}) cancer

Any suitable assay in the art can be used to detect the gene mutations of tumor cells. The high-throughput gene sequencing technology can be used to detect gene mutations.

Any suitable assay in the art can be used to assess the level of HRD, such as the use of the HRD score as described in Telli M L et al., Clin. Cancer Res., 22 (15): 3764-73 (2016), which is herein incorporated by reference in its entirety. In the present disclosure, the HRD score that is no less than 42 is regarded as a homologous recombination deficiency.

### Methods of Use

Various methods of treating diseases and disorders with the AURKA inhibitor are provided herein. Exemplary diseases and disorders that may be treated with the AURKA inhibitor disclosed herein include, but are not limited to, cancer.

In some aspects, methods of treating cancer with the AURKA inhibitor of the present disclosure are provided. Such methods comprise administering to a subject with cancer a therapeutically effective amount of a combination disclosed herein.

In some aspects, the cancer to be treated with the AURKA inhibitor disclosed hereinis selected from a hematological cancer, a lymphatic cancer, and a DNA damage repair pathway deficient cancer. In some aspects, the cancer to be treated with a combination disclosed herein is a cancer that comprises cancer cells with a mutation in a gene encoding BRCA1. In some aspects, the cancer to be treated with a combination disclosed herein is a cancer that comprises cancer cells with a mutation in a gene encoding BRCA2. In some aspects, the cancer to be treated with a combination disclosed herein is a cancer that comprises a BRCA^{Wt} cancer cells with an HRD (i.e., having an HRD score of no less than 42).

In some aspects, the cancer to be treated with a combination disclosed herein is selected from a BRCA mutant breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, or gastric cancer. In some aspects, the cancer is BRCA2 mutant cancer. For example, in some aspects, the cancer is selected from a BRCA2 mutant breast cancer, prostate cancer, colorectal cancer, small cell lung cancer, or gastric cancer. In some aspects, the cancer is BRCA1 mutant cancer. For example, in some aspects, the cancer is selected from a BRCA1 mutant breast cancer, head and neck cancer, or liver cancer. In some aspects, the cancer is a BRCA wild type cancer having a homologous recombination deficiency. For example, in some aspects, the cancer is a BRCA wild type breast cancer having a homologous recombination deficiency.

Various methods of treating cancer with the AURKA inhibitor of the present disclosure are provided herein. In some aspects, a therapeutically effective amount of a combination disclosed herein is administered to a subject with cancer.

In some aspects, such methods comprise (a) identifying a cancer in a subject to be a BRCA mutation cancer or a BRCA wild type cancer having a homologous recombination deficiency and then (b) administering a subject a therapeutically effective amount of an Aurora A inhibitor or a pharmaceutically acceptable salt, hydrate, solvate, amorphous solid, or polymorph thereof.

In some aspects, the AURKA inhibitor of the present disclosure is used to treat a cancer, wherein the cancer is a homologous-recombination deficient cancer. In some aspects, the AURKA inhibitor disclosed herein is used to treat a cancer, wherein the cancer is a BRCA1 mutant cancer. In some aspects, the AURKA inhibitor disclosed herein is used to treat a cancer, wherein the cancer is a BRCA2 mutant cancer. In some aspects, the AURKA inhibitor disclosed herein is used to treat a cancer, wherein the cancer is a BRCA1 mutant cancer and a BRCA2 mutant cancer. In some instances, the cancer is a solid tumor cancer. In some instances, the cancer is a hematological/lymphatic cancer. In some instances, the cancer is a DNA damage repair pathway deficient cancer.

In some aspects, a combination disclosed herein is used in combination with one or more additional therapeutic agents to treat cancer.

In some aspects, provided herein are the AURKA inhibitors for use as a medicament or for use in preparing a medicament, e.g., for the treatment of cancer. In some aspects, provided herein are the AURKA inhibitors for use in a method for the treatment of cancer.

### Pharmaceutical Combination Compositions

The combinations disclosed herein can be administered to a mammal in the form of raw chemicals without any other components present, or the combinations disclosed herein can also be administered to a mammal as part of a pharmaceutical composition containing the combinations disclosed herein and a suitable pharmaceutically acceptable carrier. Such a carrier can be selected from pharmaceutically acceptable excipients and auxiliaries. The term “pharmaceutically acceptable carrier” or “pharmaceutically acceptable vehicle” encompasses any of the standard pharmaceutical carriers, solvents, surfactants, or vehicles known in the art.

A pharmaceutical combination composition disclosed herein may be prepared as liquid suspensions or solutions using a liquid, such as an oil, water, an alcohol, and combinations thereof.

In the examples below, a hydrochloride salt form of 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride was used, i.e., 1-(2,3-dichlorobenzoyl)-4-[5-fluoro-6-(5-methyl-1H-pyrazol-3-ylamino)pyridin-2-yl]methyl-4-piperidinecarboxylic acid monohydrochloride, which was also known as TAS-119 or VIC-1911.

### EXAMPLES

### Cell-Derived Xenograft (CDX) Model

### Example 1

### Anti-Tumor Activity and Tolerability of VIC-1911 in the MDA-MB-436 BRCA1 Mutant Breast Cancer Model

Objective: To evaluate the anti-tumor activity and tolerability of the Aurora A inhibitor VIC-1911 at different unit doses in mice using the MDA-MB-436 BRCA1 mutant breast cancer CDX model.

Method: MDA-MB-436 cells were harvested during the logarithmic growth period and suspended in serum free media/Matrigel (1:1 volume) at the concentration of 4.3× 10⁷ cells/mL and kept on ice for tumor injection. One hundred NOD-SCID mice were inoculated with 0.1 mL tumor cell suspension (4.3× 10⁶ cell) per mouse at the right flank. Thirty days after inoculation, the tumor size reached about 170-200 mm³. Sixty mice with tumor volume from 119 mm³-204 mm³ were randomly assigned into ten groups (n=6 per group) and were dosed via oral gavage for at least 42 days according to a predetermined dosing scheme. VIC-1911 was formulated in a solution of 20% cyclodextrin+25 mM Phosphate Buffered Saline (PBS). VIC-1911 was dosed two times per day (BID) during the dosing period. The solution of 20% cyclodextrin+25 mM Phosphate Buffered Saline (PBS) without any drug active ingredient was used in the vehicle control group.

Treatment started on the day of grouping. The grouping day was marked as day 0. Body weight and tumor volume were measured twice a week until study endpoints. Compounds were administrated by oral gavage according to the scheme set forth in Table 1. The tumor volume data at 42 days were used to calculate the tumor growth inhibition. Tumor volume was calculated as mean and standard error of the mean for each treatment group.

The percentage of tumor growth inhibition (TGI %) was calculated as TGI % relative to vehicle control: TGI %=100=[(Tₙ−T₀)/(Vₙ−V₀)*100]. Tₙ is the average tumor volume of a treatment group on a given day, T₀ is the average tumor volume of the treatment group on day 0, Vₙ is the average tumor volume of the vehicle control group on the same day with Tₙ, and V₀ is the average tumor volume of the vehicle group on day 0. Data was graphed using GraphPad Prism 7.05. Statistical analysis was performed on treatment groups for tumor growth using one-way ANOVA, respectively, followed by Dunnett's multiple comparisons test.

Tolerability of each group was assessed by monitoring body weight relative to the body weight on the grouping day (day 0).

TABLE 1
Repeat dosing evaluation scheme and study results

| | | | Stop | Study | |
|---|---|---|---|---|---|
| | | | dosing | endpoints | TGI |
| | | | (days)/ | (days)/ | % on |
| No. | Compound/Group | Dose | n = 6 | n = 6 | day 42 |
| 1 | Vehicle Control* | 0 mg/kg QD | 42 | 42 | — |
| 2 | VIC-1911 | 15 mg/kg BID | 42 | 42 | 13.60 |
| 3 | VIC-1911 | 30 mg/kg BID | 42 | 42 | 25.91 |
| 4 | VIC-1911 | 60 mg/kg BID | 56 | 56 | 36.15 |
| 5 | VIC-1911 | 120 mg/kg BID | 56 | 56 | 62.87 |

*Vehicle Control: the vehicle solution without any active drug ingredient was administrated.
N = 6 refers to 6 mice used in respective groups.
BID: twice per day.
QD: once per day.

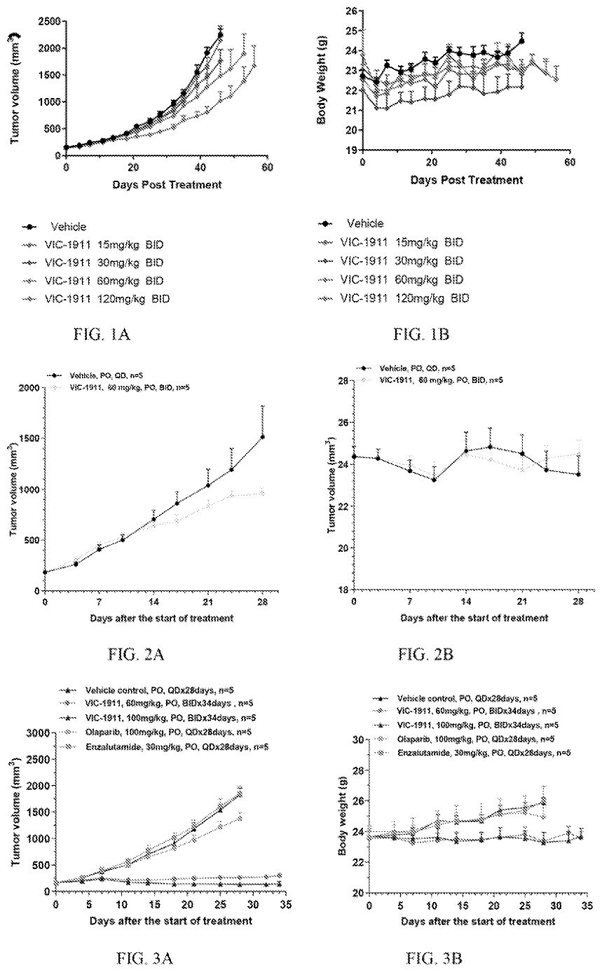

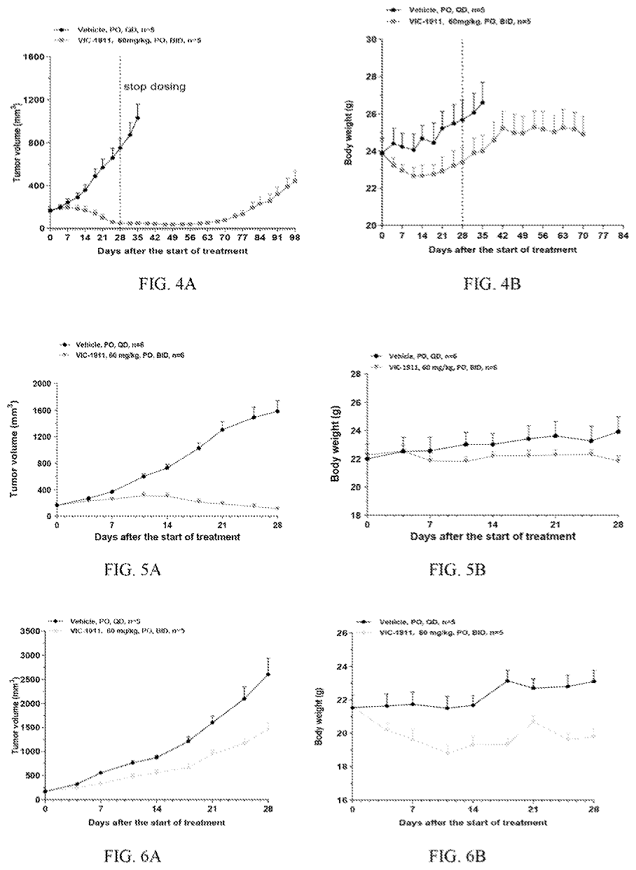

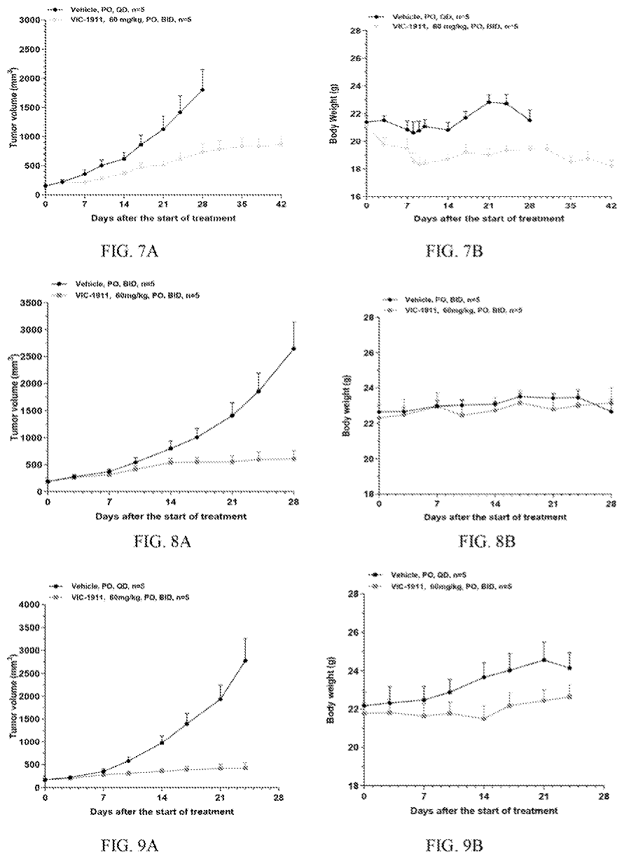

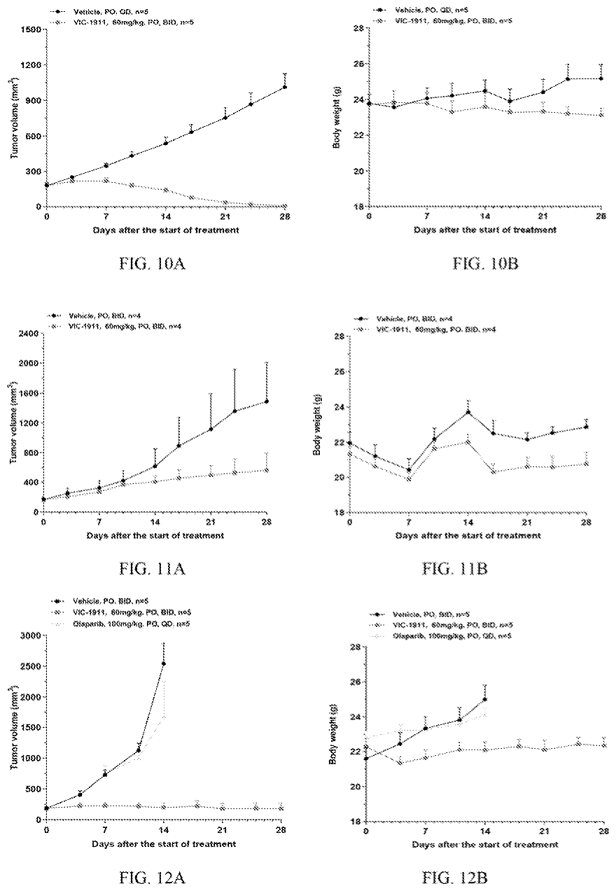

Results: The TGI % results for each group are listed in Table 1. The data in Table 1 and FIG. 1 A show that the anti-proliferation activity of the AURKA inhibitor VIC-1911 is sensitive to the administration dosage. The increased administration dosage resulted in a raised TGI % value. This indicates that the AURKA inhibitor can be effective to inhibit the cell growth of a BRCA1 mutant breast cancer. Body weight measurements as shown in FIG. 1B indicate that the AURKA inhibitor VIC-1911 was well tolerated, even in a high dosage of 120 mg/kg. Generally, the body weight loss in a range of 20% was considered to be tolerable.

### Example 2

### Anti-Tumor Activity and Tolerability of VIC-1911 in the DU145 BRCA1/2 Mutant Prostate Cancer Model

Objective: To evaluate the anti-tumor activity and tolerability of the Aurora A inhibitor VIC-1911 at 60 mg/kg in mice using the DU145 BRCA1/2 mutant prostate cancer CDX model.

Method: DU145 cells were harvested during the logarithmic growth period and suspended in serum free media/Matrigel (1:1 volume) at the concentration of 3.0×10⁷ cells/mL and kept on ice for tumor injection. One hundred NOD-SCID mice were inoculated with 0.1 mL tumor cell suspension (3.0× 10⁶ cell) per mouse at the right flank. Thirty days after inoculation, the tumor size reached about 170-200 mm³. Sixty mice with tumor volume from 170 mm³-190 mm³ were randomly assigned into ten groups (n=6 per group) and were dosed via oral gavage for at least 28 days according to a predetermined dosing scheme. VIC-1911 was formulated in a solution of 20% cyclodextrin+25 mM Phosphate Buffered Saline (PBS). The solution of 20% cyclodextrin+25 mM Phosphate Buffered Saline (PBS) without any drug active ingredient was used in the vehicle control group.

Treatment started on the day of grouping. The grouping day was marked as day 0. Body weight and tumor volume were measured twice a week until study endpoints. Compounds were administrated by oral gavage according to the scheme set forth in Table 1. The tumor volume data at 28 days were used to calculate the tumor growth inhibition. TGI % was calculated according to the method described in Example 1. Tumor volume was calculated as mean and standard error of the mean for each treatment group.

Tolerability of each group was assessed by monitoring body weight relative to the body weight on the grouping day (day 0).

Results: FIGS. 2A and 2B show the anti-tumor activity and tolerability of VIC-1911 in mice using the DU145 BRCA1 and BRCA2 mutant CDX prostate cancer model. The TGI % calculated on day 28 was 41%. The curves in FIGS. 2A and TGI % values on day 28 showed that VIC-1911 can be effective to inhibit the cell growth of the BRCA1/2 mutant prostate cancer. The body weight curve in FIG. 2B demonstrates that the VIC-1911 was well tolerated in the BRCA1/2 mutant prostate cancer.

### Example 3

Anti-Tumor Activity and Tolerability of 1-(2,3-Dichlorobenzoyl)-4-[5-Fluoro-6-(5-Methyl-1H-Pyrazol-3-Ylamino)Pyridin-2-Yl]Methyl-4-Piperidinecarboxylic Acid Monohydrochloride (VIC-1911) Relative to a PARP Inhibitor and Androgen Receptor Antagonist in the 22RV1 BRCA2 Mutant Prostate Cancer CDX Model

Objective: To evaluate the in vivo anti-tumor activity of the Aurora A inhibitor VIC-1911 relative to PARP inhibitor Olaparib and Androgen Receptor antagonist Enzalutamide in the BRCA2 mutant CDX prostate cancer model.

Method: The test method in Example 3 is almost the same as the method as described in Example 1, except that the 22RV1 BRCA2 mutant prostate cancer xenograft model was used. The 22RV1 model is a BRCA2 mutated AR* (androgen receptor) Castration Resistant Prostate Cancer model. Androgen Receptor antagonists are commonly used drugs for treating such kind of cancer. In this example, the Olaparib was formulated in a solution of 10% DMSO+10% PEG300+10% 2-Hydroxypropyl)-β-Cyclo-Dextrin (HP-β-CD). Enzalutamide, an approved Androgen Receptor antagonist, was formulated in a suitable solution for dosing. The solution of 20% cyclodextrin+25 mM Phosphate Buffered Saline (PBS) without any drug ingredient was used in the vehicle control group. The dosing scheme is listed in Table 2.

Body weight and tumor volume were measured twice per week from day 0 to study endpoints. The average tumor value on day 14 was taken to calculate the TGI % for each group. The TGI % was calculated according to the method described in Example 1.

TABLE 2
Repeat dosing evaluation scheme and study results

| | | | Study | |
|---|---|---|---|---|
| | | | endpoints | TGI % on |
| No. | Compound/Group | Dose | (days)/n = 5 | day 28 |
| 1 | Vehicle Control | 0 mg/kg QD | 28 | — |
| 2 | VIC-1911 | 60 mg/kg BID | 34 | 94 |
| 3 | VIC-1911 | 100 mg/kg BID | 34 | 102 |
| 4 | Olaparib | 100 mg/kg QD | 28 | 27 |
| 5 | Enzalutamide | 30 mg/kg QD | 28 | −0.7 |

n = 5: each group contained 5 mice.

Results: As shown in Table 2 and FIG. 3A, the 60 mg/kg group and 100 mg/kg group for VIC-1911 each achieved a high tumor growth inhibition over 90%. This indicates the effectiveness of VIC-1911 to a BRCA2 mutant prostate cancer. As compared to the curves and TGI % values of the Olaparib and Enzalutamide groups, the VIC-1911 group exhibited an obviously more potent efficacy. Moreover, curves in FIG. 3B show that VIC-1911 was also well tolerated from the minor change of body weight.

### Patient Derived Xenograft (PDX) Models

### Example 4

### Anti-Tumor Activity and Tolerability of 1-(2,3-Dichlorobenzoyl)-4-[5-Fluoro-6-(5-Methyl-1H-Pyrazol-3-Ylamino)Pyridin-2-Yl]Methyl-4-Piperidinecarboxylic Acid Monohydrochloride (VIC-1911) in Patient Derived Xenograft (PDX) Models

Objective: To evaluate the in vivo anti-tumor activity and tolerability of the Aurora A inhibitor VIC-1911 in a panel of different BRCA mutant PDX models.

### Method:

### a) Generation of the PDX Model

Each selected PDX model was originally established as follows. The surgically resected clinical sample from a suitable patient was implanted and grown in nude mice defined as passage 0 (PO). The next passage implanted and grown from PO tumor was defined as passage 1 (P1), and so on during continual implantation in mice.

### b) Tumor Implantation and Animal Grouping

Each mouse was implanted subcutaneously at the right flank with the tumor slices (20˜30 mm³) from a selected PDX model for tumor development. Treatments were started when the average tumor size reached approximately 150-200 mm³. Several mice were chosen and randomly assigned into a number of groups. The grouping day was marked as the day 0.

Treatments were started on the day of the grouping. VIC-1911 was formulated in a solution of 20% cyclodextrin+25 mM Phosphate Buffered Saline (PBS), and orally dosed at a unit dosage of 60 mg/kg twice a day. The solution of 20% cyclodextrin+25 mM Phosphate Buffered Saline (PBS) without any drug ingredient was used in vehicle control group. Mice in the control group were dosed once per day.

The PDX models used and the treating protocols are listed in the Table 3.

Body weight and tumor volume were measured twice per week from day 0 to study endpoints. The tumor volume on a selected day D was taken to calculate the TGI % according to the method as described in Example 1.

TABLE 3
PDX model information and study results

| No. | Model name | Mutant type | Cancer type | TGI %(D) |
|---|---|---|---|---|
| 1 | BR-05-0014E | BRCA2 | Breast cancer | 120(28) |
| 2 | BR-05-0028 | BRCA1 | Breast cancer | 104(28) |
| 3 | OV-10-0060 | BRCA2 | Ovarian cancer | 47(28) |
| 4 | OV-10-0079 | BRCA1/2 | Ovarian cancer | 65(28) |
| 5 | HN-13-0032 | BRCA1 | Head and neck | 83(28) |
| | | | cancer | |
| 6 | LU-01-1388 | BRCA1/2 | Small cell lung | 90(24) |
| | | | cancer | |
| 7 | LU-01-1116 | BRCA2 | Small cell lung | 121(28) |
| | | | cancer | |
| 8 | ST-02-0360 | BRCA2 | Gastric cancer | 70(28) |

D refers to the day when the TGI was calculated with tumor volume.

### Results:

### BR-05-0014E Model

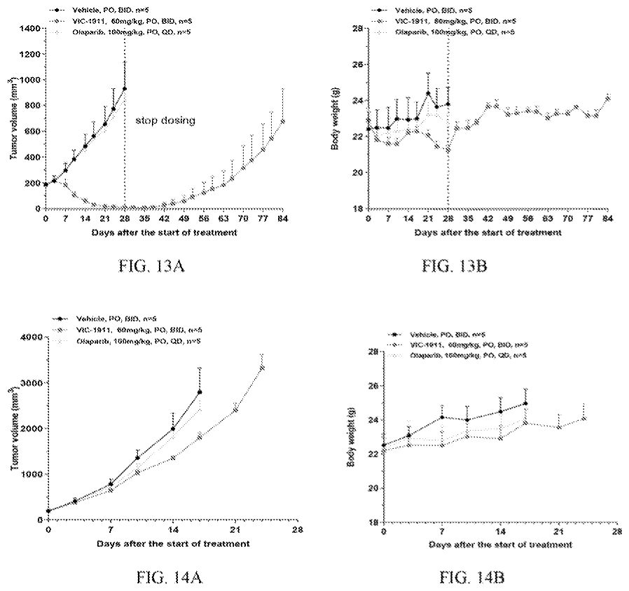

FIGS. 4A and 4B show the anti-tumor activity and tolerability of VIC-1911 in mice using the BR-05-0014E BRCA2 mutant PDX models. In the BR-05-0014E PDX model, the drug dosing was stopped on day 28, but the tumor volume and body weight measurements were still kept until day 98 for evaluation of the rebounding of tumors. Tumor volume obtained on day 28 was used to calculated TGI %. The equation n= # in the Figures refers to the number of mice used in respective groups.

The curves in FIGS. 4A and TGI % (120% on day 28) in Table 3 exhibit the excellent anti-tumor activity of VIC-1911 in the BRCA2 mutant breast cancer. The curves in FIG. 4A also indicates that VIC-1911 can even lead to the tumor regression in this BRCA2 mutant breast cancer PDX model.

After the discontinuation of dosing, the tumor volume from day 28 to day 63 was almost kept at the same level. That is, no rebounding of tumors was observed for up to 35 days. Such a result indicates the superior performance of VIC-1911 in inhibiting the rebounding of tumors. The body weight curve in FIG. 4B demonstrates the good tolerability of VIC-1911.

### BR-05-0028 Model

FIGS. 5A and 5B show the anti-tumor activity and tolerability of VIC-1911 in mice using the BR-05-0028 BRCA1 mutant PDX breast cancer models. The curves in FIG. 5A and TGI % (104% on day 28) in Table 3 exhibit the excellent anti-tumor activity of VIC-1911 in the BRCA1 mutant breast cancer. The body weight curve in FIG. 5B also demonstrates the satisfactory tolerability of VIC-1911 in this model.

### OV-10-0060 Model

FIGS. 6A and 6B show the anti-tumor activity and tolerability of VIC-1911 in mice using the OV-10-0060 BRCA2 mutant PDX ovarian cancer model. The curves in FIG. 6A and TGI % (47% on day) in Table 3 exhibit that VIC-1911 can be effective to inhibit the tumor growth of the BRCA2 mutant ovarian cancer. The body weight curve in FIG. 6B demonstrates that the VIC-1911 was tolerated in the BRCA2 mutant ovarian cancer.

### OV-10-0079 Model

FIGS. 7A and 7B show the anti-tumor activity and tolerability of VIC-1911 in mice using the OV-10-0079 BRCA1 and BRCA 2 mutant PDX ovarian cancer model. The curves in FIG. 7A and TGI % (65% on day 28) in Table 3 exhibit that VIC-1911 can be effective to inhibit the tumor growth of the BRCA1/2 mutant ovarian cancer. The body weight curve in FIG. 7B demonstrates that the VIC-1911 was tolerated in the BRCA1/2 mutant ovarian cancer.

### HN-13-0032 Model

FIGS. 8A and 8B show the anti-tumor activity and tolerability of VIC-1911 in mice using the HN-13-0032 BRCA1 mutant PDX head and neck cancer model. The curves in FIG. 8A and TGI % (83% on day 28) in Table 3 exhibit the excellent anti-tumor activity of VIC-1911 in the BRCA1 mutant head and neck cancer. The body weight curve in FIG. 8B also demonstrates the satisfactory tolerability of VIC-1911 in the BRCA1 mutant head and neck cancer model.

### LU-01-1388 Model

FIGS. 9A and 9B show the anti-tumor activity and tolerability of VIC-1911 in mice using the LU-01-1388 BRCA1 and BRCA2 mutant PDX small cell lung cancer (SCLC) model. The curves in FIG. 9A and TGI % (90% on day 24) in Table 3 exhibit the excellent anti-tumor activity of VIC-1911 in the BRCA1/2 mutant SCLC. The body weight curve in FIG. 3B also demonstrates the satisfactory tolerability of VIC-1911 in this model.

### LU-01-1116 Model

FIGS. 10A and 10B show the anti-tumor activity and tolerability of VIC-1911 in mice using the LU-01-1116 BRCA2 mutant PDX small cell lung cancer model. The curves in FIG. 10A and TGI % (121% on day 28) in Table 3 exhibit the excellent anti-tumor activity of VIC-1911 in the BRCA2 mutant SCLC. The curves in FIG. 10A also indicate that VIC-1911 can even lead to the complete regression of tumor in this BRCA2 mutant SCLC cancer PDX model. The body weight curve in FIG. 10B also demonstrates the satisfactory tolerability of VIC-1911 in this model.

### ST-02-0360 Model

FIGS. 11A and 11B show the anti-tumor activity and tolerability of VIC-1911 in mice using the ST-02-0360 BRCA2 mutant PDX gastric cancer model. The curves in FIGS. 11A and TGI % (70% on day 28) in Table 3 exhibit the good anti-tumor activity of VIC-1911 in the BRCA2 mutant gastric cancer. The body weight curve in FIG. 11B also demonstrates the good tolerability of VIC-1911 in the BRCA2 mutant gastric cancer model.

### Example 5

Anti-Tumor Activity and Tolerability of 1-(2,3-Dichlorobenzoyl)-4-[5-Fluoro-6-(5-Methyl-1H-Pyrazol-3-Ylamino)Pyridin-2-Yl]Methyl-4-Piperidinecarboxylic Acid Monohydrochloride (VIC-1911) and Comparison to a PARP Inhibitor in the BR-05-0020E BRCA Wild Type Breast Cancer PDX Model

Objective: To evaluate the in vivo anti-tumor activity of the Aurora A inhibitor VIC-1911 and the comparison between VIC-1911 and Olaparib in a cancer with an HRD. In this example, the BRCA wild type (BRCA^{Wt}) breast PDX model with an HRD score of 64.83 was used. The HRD score for this model was evaluated according to the method described in Telli M L et al., Clin. Cancer Res., 22 (15): 3764-73 (2016).

Method: The test method in Example 5 is the same as the method as described in Example 4, except that the BR-05-0020E BRCA^{Wt} breast cancer PDX model was used. In this example, Olaparib was formulated in a solution of 10% DMSO+10% PEG300+10% 2-Hydroxypropyl)-β-Cyclo-Dextrin (HP-β-CD). The solution of 20% cyclodextrin+25 mM Phosphate Buffered Saline (PBS) without any drug ingredient was used in vehicle control group. The dosing scheme is listed in Table 4.

Body weight and tumor volume were measured twice per week from day 0 to study endpoints. The average tumor value on day 14 was taken to calculate the TGI % for each group. The TGI % was calculated according to the method described in Example 1.

TABLE 4
Repeat dosing evaluation scheme and study results

| | | | Study | |
|---|---|---|---|---|
| | | | endpoints | TGI % on |
| No. | Compound/Group | Dose | (days)/n = 5 | day 14 |
| 1 | Vehicle Control | 0 mg/kg QD | 14 | — |
| 2 | VIC-1911 | 60 mg/kg BID | 28 | 99 |
| 3 | Olaparib | 100 mg/kg QD | 14 | 36 |

n = 5: each group contained 5 mice.

Results: As shown in FIG. 12A, the tumor volume for the VIC-1911 group exhibited a great reduction relative to the tumor volume of vehicle control group from the early 4 days. Besides, the tumor volume of VIC-1911 group was basically a horizontal line from day 0 to day 28, resulting in a TGI % of 99% at day 14. These results demonstrate the excellent efficacy of VIC-1911 in treating the BRCA^{Wt} cancer with an HRD. FIG. 12A also shows the anti-tumor activity of Olaparib, in which the tumor volume of the Olaparib group was almost consistent with the tumor volume of vehicle control group from day 0 to day 11, and finally exhibited a TGI % of 36 at day 14. By the comparison between the VIC-1911 and Olaparib groups, it can be seen that VIC-1911 can be more effective relative to the Olaparib in treating the BRCA^{Wt} cancer with an HRD. FIG. 12B shows a slight body weight loss for the VIC-1911 group, indicating the good tolerability of VIC-1911.

### Example 6

Anti-Tumor Activity and Tolerability of 1-(2,3-Dichlorobenzoyl)-4-[5-Fluoro-6-(5-Methyl-1H-Pyrazol-3-Ylamino)Pyridin-2-Yl]Methyl-4-Piperidinecarboxylic Acid Monohydrochloride (VIC-1911) and Comparison to a PARP Inhibitor in the LI-03-0014 BRCA1 Mutant Liver Cancer PDX Model

Objective: To evaluate the in vivo anti-tumor activity of the Aurora A inhibitor VIC-1911 and its comparison relative to that of Olaparib using the LI-03-0014 BRCA1 mutant liver cancer PDX model.

Method: The test method in Example 6 is the same as the method as described in Example 4, except that the LI-03-0014 triple negative BRCA1 mutant liver cancer PDX model with an HRD score of 68.93 was used. In this example, Olaparib was formulated in a solution of 10% DMSO+10% PEG300+10% 2-Hydroxypropyl)-β-Cyclo-Dextrin (HP-β-CD). The solution of 20% cyclodextrin+25 mM Phosphate Buffered Saline (PBS) without any drug ingredient was used in the vehicle control group. The dosing scheme is listed in Table 5.

Body weight and tumor volume were measured twice per week from day 0 to study endpoints. The average tumor value on day 28 was taken to calculate the TGI % for each group. The TGI % was calculated according to the method described in Example 1.

TABLE 5
Repeat dosing evaluation scheme and study results

| | | | Stop | Study | |
|---|---|---|---|---|---|
| | | | dosing | endpoints | TGI |
| | | | (days)/ | (days)/ | % on |
| No. | Compound/Group | Dose | n = 5 | n = 5 | day 28 |
| 1 | Vehicle Control | 0 mg/kg QD | 28 | 28 | — |
| 2 | VIC-1911 | 60 mg/kg BID | 28 | 84 | 121 |
| 3 | Olaparib | 100 mg/kg QD | 28 | 28 | 11 |

n = 5: each group contained 5 mice.

Results: As shown in Table 5 and FIG. 13A, the TGI % value of the VIC-1911 group at the unit dosage of 60 mg/kg was 121%, which demonstrates significant anti-tumor activity of VIC-1911 in the BRCA1 mutant liver cancer PDX model. FIG. 13A also shows the anti-tumor activity of Olaparib, in which the tumor volume of the Olaparib group was almost consistent with the tumor volume of the vehicle control group from day 0 to day 28, and finally exhibited a TGI % of 11 at day 28. The much higher TGI % (121%) of the VIC-1911 group relative to the TGI % (11%) of the Olaparib group indicates the remarkable advantage of VIC-1911 over Olaparib in the treatment of a BRCA1 mutant liver cancer.

FIG. 13A also shows the tumor volume of the VIC-1911 group as a function of time after the discontinuation of drug dosing. As also shown in FIG. 13A, after the discontinuation of drug dosing, the tumor volume for the VIC-1911 group maintained a low level from day 28 to day 45. Such a result indicates that VIC-1911 can also be effective to inhibit the rebounding of tumor cells.

FIG. 13B shows a slight body weight loss for the VIC-1911 group, indicating the good tolerability of VIC-1911.

### Example 7

Anti-Tumor Activity and Tolerability of 1-(2,3-Dichlorobenzoyl)-4-[5-Fluoro-6-(5-Methyl-1H-Pyrazol-3-Ylamino)Pyridin-2-Yl]Methyl-4-Piperidinecarboxylic Acid Monohydrochloride (VIC-1911) and Comparison to a PARP Inhibitor in the CO-04-0003 BRCA2 Mutant Colorectal Cancer PDX Model

Objective: To evaluate the in vivo anti-tumor activity of the Aurora A inhibitor VIC-1911 and the comparison between VIC-1911 and Olaparib using the CO-04-0003 BRCA2 mutant colorectal cancer PDX model.

Method: The test method in Example 7 is the same as the method as described in Example 4, except that the CO-04-0003 BRCA2 mutant colorectal cancer PDX model was used. The dosing scheme is listed in Table 6. The data on day 17 was taken to calculate the TGI % for each group.

TABLE 6
Repeat dosing evaluation scheme and study results

| | | | Study | |
|---|---|---|---|---|
| | | | endpoints | TGI % on |
| No. | Compound/Group | Dose | (days)/n = 5 | day 17 |
| 1 | Vehicle Control* | 0 mg/kg QD | 17 | — |
| 2 | VIC-1911 | 60 mg/kg BID | 24 | 38 |
| 3 | Olaparib | 100 mg/kg QD | 17 | 14 |

*Vehicle Control: the vehicle solution without any active drug ingredient was administrated.

Results: The TGI % results for each group are listed in Table 6. As shown in Table 6 and FIG. 14A, VIC-1911 can be effective to inhibit the cell growth of a BRCA2 mutant colorectal cancer. The TGI % value (38%) of the VIC-1911 group at 60 mg/kg was higher than the TGI % value (14%) of the Olaparib group at 100 mg/kg. Such a result indicates a more effective inhibition of VIC-1911 on cell growth compared to Olaparib.

Body weight measurements indicate that there was no significant difference in tolerability between the VIC-1911 group and the Olaparib group, as shown in FIG. 14B.

### CONCLUSION

The AURKA inhibitor (such as VIC-1911) exhibit anti-tumor activity in BRCA mutant cancer xenograft models and the BRCA^{Wt} cancer with an HRD. This indicate that an AURKA inhibitor can be effective to inhibit the cell growth of the BRCA mutant cancer and the BRCA^{Wt} cancer with an HRD.

In some embodiments (such as in the BRCA2 mutant breast cancer model), the AURKA inhibitor (such as VIC-1911) can induce the tumor to regress, and even inhibit the rebounding of tumors for over one month after discontinuation of drug administration.

In some embodiments (such as in the BRCA2 mutant SCLC), the AURKA inhibitor (such as VIC-1911) can induce the tumor to regress, and even to completely regress.

In some embodiments (such as Examples 3, 5-7), the AURKA inhibitor (such as VIC-1911) exhibit remarkable advantages over the PARP inhibitor Olaparib in the treatment of BRCA mutant cancers and BRCA^{Wt} cancers with HRDs.

In some embodiments (such as Example 3), the AURKA inhibitor (such as VIC-1911) exhibit remarkable advantages over the androgen receptor antagonist Enzalutamide in the treatment of the BRCA2 mutant prostate cancer.

### EQUIVALENTS

While the present invention has been described in conjunction with the specific embodiments set forth above, many alternatives, modifications, and other variations thereof will be apparent to those of ordinary skill in the art. All such alternatives, modifications, and variations are intended to fall within the spirit and scope of the present invention. All of the patent applications and non-patent publications referred to in this specification are incorporated herein by reference, in their entirety. Aspects of the embodiments can be modified if necessary to employ concepts of the various patents, applications, and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating a cancer in a subject in need thereof, wherein the method comprises:
a) identifying the cancer to be a BRCA mutation cancer or a BRCA wild type cancer with a homologous recombination deficiency (HRD); and
b) administering to the subject a therapeutically effective amount of an Aurora A inhibitor or a pharmaceutically acceptable salt, hydrate, solvate, amorphous solid, or polymorph thereof.

2. The method according to claim 1, wherein the cancer is a BRCA mutant breast cancer, ovarian cancer, peritoneal cancer, fallopian tube cancer, prostate cancer, colorectal cancer, head and neck cancer, liver cancer, non-small cell lung cancer (NSCLC), small cell lung cancer (SCLC), pancreatic cancer, glioma, or gastric cancer.

3. The method according to claim 1, wherein the cancer is a BRCA2 mutant cancer.

4. The method according to claim 3, wherein the cancer is a BRCA2 mutant cancer having a homologous recombination deficiency.

5. The method according to claim 1, wherein the cancer is a BRCA2 mutant breast cancer, prostate cancer, colorectal cancer, small cell lung cancer or gastric cancer.

6. The method according to claim 1, wherein the cancer is a BRCA2 mutant breast cancer.

7. The method according to claim 1, wherein the cancer is a BRCA2 mutant prostate cancer.

8. The method according to claim 1, wherein the cancer is a BRCA2 mutant small cell lung cancer.

9. The method according to claim 1, wherein the cancer is a BRCA2 mutant gastric cancer.

10. The method according to claim 4 or 6, wherein the cancer is a BRCA2 mutant breast cancer having a homologous recombination deficiency.

11. The method according to claim 1, wherein the cancer is a BRCA1 mutant cancer.

12. The method according to claim 1, wherein the cancer is a BRCA1 mutant breast cancer, head and neck cancer, or liver cancer.

13. The method according to claim 1, wherein the cancer is a BRCA1 mutant breast cancer.

14. The method according to claim 1, wherein the cancer is a BRCA1 mutant head and neck cancer.

15. The method according to claim 1, wherein the cancer is a BRCA1 mutant liver cancer.

16. The method according to claim 1, wherein the cancer is a BRCA1 and BRCA2 mutant cancer.

17. The method according to claim 1, wherein the cancer is a BRCA1 and BRCA2 mutant ovarian cancer, prostate cancer, or small cell lung cancer.

18. The method according to claim 1, wherein the cancer is a BRCA1 and BRCA2 mutant ovarian cancer.

19. The method according to claim 1, wherein the cancer is a BRCA1 and BRCA2 mutant small cell lung cancer.

20. The method according to claim 1, wherein the cancer is a BRCA wild type cancer having a homologous recombination deficiency.

21. The method according to claim 1, wherein the cancer is a BRCA wild type breast cancer having a homologous recombination deficiency.

22. The method according to any one of the proceeding claims, wherein the Aurora A inhibitor comprises a compound, which is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof.

23. The method according to any one of the proceeding claims, wherein the Aurora A inhibitor comprises a compound, which is 1-(2,3-dichlorobenzoyl)-4-[5-fluoro-6-(5-methyl-1H-pyrazol-3-ylamino)pyridin-2-yl]methyl-4-piperidine carboxylic acid monohydrochloride.

24. The method according to to any one of preceeding claims, wherein the Aurora A inhibitor is orally administered twice per day at a unit dose ranging from 15 mg/kg to 200 mg/kg.

25. The method according to any one of preceeding claims, wherein the Aurora A inhibitor is orally administered twice per day at a unit dose ranging from 20 mg/kg to 150 mg/kg.

26. The method according to any one of preceeding claims, wherein the Aurora A inhibitor is orally administered twice per day at a unit dose ranging from 60 mg/kg to 120 mg/kg.

27. The method according to any one of preceeding claims, wherein the Aurora A inhibitor is administered as a composition comprising the Aurora A inhibitor and a pharmaceutically acceptable carrier, diluent, or excipient.

28. Use of an Aurora A inhibitor in the treatment of a BRCA mutation cancer or a BRCA wild type cancer with an HRD.

29. The use according to claim 28, wherein the Aurora A inhibitor comprises a compound, which is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof.

30. The use according to claim 28 or 29, wherein the Aurora A inhibitor comprises a compound, which is 1-(2,3-dichlorobenzoyl)-4-[5-fluoro-6-(5-methyl-1H-pyrazol-3-ylamino)pyridin-2-yl]methyl-4-piperidine carboxylic acid monohydrochloride.

31. A compound, which is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof, for use in the preparation of a medicament for treating a BRCA mutant cancer or a BRCA wild type cancer having an HRD.

32. A compound, which is 1-(2,3-dichlorobenzoyl)-4-[5-fluoro-6-(5-methyl-1H-pyrazol-3-ylamino)pyridin-2-yl]methyl-4-piperidine carboxylic acid, or a pharmaceutically acceptable salt, hydrate, solvate, amorphous solid, or polymorph thereof, for use in the treatment of a BRCA mutant cancer or a BRCA wild type cancer having an HRD.
